(19) United States
(12) Patent Application Publication
(10) Pub. No.: US 2021/0354285 A1
(43) Pub. Date: Nov. 18, 2021

(54) MECHANICAL COUPLING TO JOIN TWO COLLABORATIVE ROBOTS TOGETHER FOR MEANS OF CALIBRATION

(71) Applicant: Activ Surgical, Inc., Boston, MA (US)

(72) Inventors: Vasiliy Evgenyevich Buharin, Boston, MA (US); Emanuel DeMaio, Boston, MA (US); Liam O'Shea, Boston, MA (US); Jessica Hanley, Boston, MA (US)

(21) Appl. No.: 17/338,030

(22) Filed: Jun. 3, 2021

Related U.S. Application Data
(63) Continuation of application No. PCT/US2019/065056, filed on Dec. 6, 2019.
(60) Provisional application No. 62/776,842, filed on Dec. 7, 2018.

(51) Int. Cl. B25J 9/00; A61B 34/30; B25J 9/16

(52) U.S. Cl. CPC B25J 9/009; A61B 2034/302; B25J 9/1692; A61B 34/30

(57) ABSTRACT

Systems and methods for mechanical coupling and calibration of two fixed-base robotic arms are disclosed. In particular, a first robotic arm is affixed to a first base at a proximal end and has a first coupling at a distal end and a second robotic arm is affixed to a second base at a proximal end and has a second coupling at a distal end. The first coupling is releasably coupled to a second coupling via a locking mechanism to prevent relative motion between the first and second couplings. Three-dimensional positional data is collected for the distal ends of the first robotic arm and the second robotic arm in one or more positions. A calibration value is determined from the three-dimensional positional data. The calibration value may be a calibration matrix determined by a least mean squares method.

### CROSS-REFERENCE TO RELATED APPLICATION

This application is a continuation application of International Application No. PCT/US2019/065056, filed on Dec. 6, 2019, which application claims the benefit of U.S. Provisional Application No. 62/776,842, filed Dec. 7, 2018, which applications are incorporated herein by reference in their entirety for all purposes.

### BACKGROUND

Embodiments of the present disclosure generally relate to calibration of three-dimensional position information between two fixed-base robotic arms. In particular, the present disclosure describes a mechanical coupling that allows the arms of two fixed-base robotic arms to be calibrated and methods for calibrating two fixed base robotic arms after coupling the two arms together.

### BRIEF SUMMARY

According to embodiments of the present disclosure, systems for, methods for, and computer program products for calibration of two fixed-base robotic arms are provided. In various embodiments, a system includes a first robotic arm having a proximal end and a distal end. The proximal end of the first robotic arm is fixed to a first base. The system further includes a second robotic arm having a proximal end and a distal end. The proximal end of the second robotic arm is fixed to a second base. A first coupling is disposed at the distal end of the first robotic arm. The first coupling has a first flange and a protrusion extending from the flange. A second coupling is disposed at the distal end of the second robotic arm, the second coupling having a second flange and a recess in the second flange having a shape corresponding to a shape of the protrusion. A locking mechanism releasably couples the first flange and the second flange together such that the distal end of the first robotic arm and the distal end of the second robotic arm move together. The first robotic arm and second robotic are configured to be calibrated to one another when the first coupling is coupled to the second coupling by collecting positional data in at least a first and a second coupled position and determining therefrom a calibration value.

In various embodiments, the locking mechanism includes a collar. In various embodiments, the locking mechanism includes a fastener. In various embodiments, the protrusion has rotational symmetry about an axis normal to the first flange. In various embodiments, a shape of the protrusion is selected from the group consisting of: square, rectangle, triangle, and circle. In various embodiments, the protrusion has rotational asymmetry about an axis normal to the first flange. In various embodiments, the recess has rotational symmetry about an axis normal to the second flange. In various embodiments, a shape of the recess is selected from the group consisting of: square, rectangle, triangle, and circle. In various embodiments, the recess has rotational asymmetry about an axis normal to the second flange. In various embodiments, the second flange includes two or more recesses. In various embodiments, the first flange includes two or more protrusions.

In various embodiments, a method for calibrating two robotic arms includes providing a first robotic arm having a proximal end and a distal end and a second robotic arm having a proximal end and a distal end. The proximal end of the first robotic arm is fixed to a first base and the proximal end of the second robotic arm is fixed to a second base. The distal end of the first robotic arm is releasably coupled to the distal end of the second robotic arm via a coupling at a first coupled position. First positional data for the distal end of the first robotic arm is collected at the first coupled position. Second positional data for the distal end of the second robotic arm is collected at the first coupled position. A calibration value based at least on the first positional data and the second positional data is determined. In various embodiments, the calibration value may be a calibration matrix determined by a least mean squares method.

In various embodiments, after collecting the first positional data and the second positional data, the first and second robotic arms are moved to a second coupled position, third positional data is collected for the distal end of the first robotic arm while in the second coupled position, fourth positional data is collected for the distal end of the second robotic arm while in the second coupled position, and the calibration value is determined using the third and fourth positional data.

In various embodiments, after collecting the first positional data and the second positional data, the distal ends of the first and second robotic arms are moved to a second coupled position, third positional data is collected for the distal end of the first robotic arm while in the second coupled position, fourth positional data is collected for the distal end of the second robotic arm while in the second coupled position, and the calibration value is applied to each of the third and fourth positional data.

In various embodiments, the calibration value includes a calibration matrix. In various embodiments, the calibration matrix is determined by a least mean squares method. In various embodiments, the calibration matrix is determined by a least squares method. In various embodiments, the calibration matrix is determined by a Kalman filter. In various embodiments, the first robotic arm and the second robotic arm are configured to perform a medical procedure. In various embodiments, the medical procedure is a gastric bypass surgery. In various embodiments, the first positional data and the second positional data each includes three-dimensional data. In various embodiments, collecting first positional data and collecting second positional data each includes continuous recording of data.

In various embodiments, a computer program product is provided for calibrating two fixed-base robotic arms. The computer program product includes a computer readable storage medium having program instructions embodied therewith and the program instructions are executable by a processor to cause the processor to perform a method including collecting first positional data from a distal end of a first robotic arm releasably coupled to a distal end of a second robotic arm at a first coupled position. Second positional data is collected from the distal end of the second robotic arm at the first coupled position. A calibration value based at least on the first positional value and the second positional value is determined. In various embodiments, the calibration value may be a calibration matrix determined by a least mean squares method.

In various embodiments, after collecting the first positional data and the second positional data, the first and second robotic arms are moved to a second coupled position, third positional data is collected for the distal end of the first robotic arm while in the second coupled position, fourth positional data is collected for the distal end of the second robotic arm while in the second coupled position, and the calibration value is determined using the third and fourth positional data.

In various embodiments, after collecting the first positional data and the second positional data, the distal ends of the first and second robotic arms are moved to a second coupled position, third positional data is collected for the distal end of the first robotic arm while in the second coupled position, fourth positional data is collected for the distal end of the second robotic arm while in the second coupled position, and the calibration value is applied to each of the third and fourth positional data.

In various embodiments, the calibration value includes a calibration matrix. In various embodiments, the calibration matrix is determined by a least mean squares method. In various embodiments, the calibration matrix is determined by a least squares method. In various embodiments, the calibration matrix is determined by a Kalman filter. In various embodiments, the first robotic arm and the second robotic arm are configured to perform a medical procedure. In various embodiments, the medical procedure is a gastric bypass surgery. In various embodiments, the first positional data and the second positional data each includes three-dimensional data. In various embodiments, collecting first positional data and collecting second positional data each includes continuous recording of data.

### BRIEF DESCRIPTION OF THE DRAWINGS

FIG. 1 illustrates an exemplary coupling for a fixed-base robotic arm according to an embodiment of the present disclosure.

FIGS. 2A-2E illustrate steps to couple together two exemplary couplings for fixed-base robotic arms according to an embodiment of the present disclosure.

FIG. 3 illustrates two exemplary fixed-base robotic arms coupled to one another according to an embodiment of the present disclosure.

FIGS. 4A-4B illustrate two exemplary couplings for fixed-base robotic arms according to an embodiment of the present disclosure.

FIG. 5 illustrates exemplary couplings for a fixed-base robotic arm according to an embodiment of the present disclosure.

FIG. 6 illustrates exemplary couplings for a fixed-base robotic arm according to an embodiment of the present disclosure.

FIG. 7 illustrates a flowchart of a method for calibrating two fixed-base robotic arms according to an embodiment of the present disclosure.

FIG. 8 depicts an exemplary computing node according to various embodiments of the present disclosure.

### DETAILED DESCRIPTION

Many surgical maneuvers (e.g., suturing, cutting, and/or folding) require highly dexterous and highly accurate motion of surgical tools to achieve a satisfactory surgical outcome. These surgical maneuvers may require more than one robotic arm to adequately perform the particular maneuver, for example, where one robotic arm holds a tissue while the other robotic arm sutures or cuts the tissue. In fully automated robotic surgical procedures using two or more robots that are cooperating to perform a surgical maneuver, each of the robots may operate within the same three-dimensional coordinate system to provide accurate collaborative motions (e.g., hand-offs) and collision prevention/detection between the robots. A calibration map may be provided to enable the robots to operate within the same coordinate system, thereby enabling the robots to work together.

In various embodiments, one option for calibrating two or more robots in a robotic system involves placing each of the robots in predetermined locations having known coordinates in a 3-dimensional coordinate system. In various embodiments, program code for each robot is adjusted prior to using the robotic system to account for any movement within the operative field. However, this method is not completely accurate as knowledge of robot position may not be absolute. For example, any error in the placement of the robots may result in a suboptimal calibration, which may cause imprecise motion of the robots and/or collision between robots. In fields where room for error is very small, such as in robotic-assisted surgery, each collaborative robot must be accurately and precisely calibrated to minimize the risk of adverse events.

Accordingly, a need exists for a system and method to calibrate two or more fixed-based robotic arms thereby enabling accurate surgical maneuvers and cooperation between the two or more robotic arms to improve robotic-assisted surgery.

Embodiments of the present disclosure generally relate to calibration of three-dimensional position information between two fixed-base robotic arms. In particular, the present disclosure describes a mechanical coupling that allows the arms of two fixed-base robotic arms to be calibrated and methods for calibrating two fixed base robotic arms after coupling the two arms together. While the present disclosure generally focuses on calibrating the three-dimensional position with respect to two or more automated surgical robots, the systems, methods, and computer program products are suitable for use in other fields that employ collaborative robots, such as manufacturing, consumer robotics, or other autonomous robots.

The two or more robotic arms may perform a surgical maneuver or other collaborative maneuver. In various embodiments, the collaborative maneuver may be a hand-off In various embodiments, a hand-off may involve transferring an object being held by a first robotic arm to a second robotic arm, thus freeing up the first robotic arm to perform other functions. In various embodiments, the collaborative maneuver may involve any suitable biological tissue. For example, the biological tissue may be an internal bodily tissue, such as esophageal tissue, stomach tissue, small/large intestinal tissue, and/or muscular tissue. In other embodiments, the object may be external tissue, such as dermal tissue on the abdomen, back, arm, leg, or any other external body part. Moreover, the biological tissue may be a bone, internal organ, or other internal bodily structure. The systems and methods of the present disclosure would similarly work for animals in veterinary applications.

A system for calibrating two fixed-base robotic arms may include a first robotic arm having a proximal end and a distal end and a second robotic arm having a proximal end and a distal end. The proximal end of the first robotic arm is fixed to a first base and the proximal end of the second robotic arm is fixed to a second base. A first coupling may be disposed at the distal end of the first robotic arm and a second coupling may be disposed at the distal end of the second robotic arm. The first coupling may have a first flange and one or more protrusion and the second coupling may have a second flange and a recess in the second flange having a shape corresponding to a shape of the protrusion of the first coupling. A locking mechanism may releasably couple the first flange and the second flange. In various embodiments, the locking mechanism may be a collar or clamp. In various embodiments, the locking mechanism may include a wingnut screw.

In various embodiments, the protrusion may include any suitable shape for engaging with the recess as is known in the art. For example, the protrusion may include a taper where the protrusion tapers from a wider diameter to a narrower diameter. In this example, the recess may include a corresponding taper such that the tapered protrusion may matingly engage the recess. In various embodiments, more than one protrusion may be provided on the first coupling. In various embodiments, more than one recess may be provided on the second coupling. In various embodiments, the protrusion(s) and recess(es) may be arranged such that relative motion between the distal ends of the robotic arms is prevented when the protrusion(s) matingly engages the corresponding recess(es).

In various embodiments, a method for calibrating two robotic arms includes providing a first robotic arm and a second robotic arm. Each robotic arm is fixed to a base (which may be the same base or different bases) at their respective proximal-most end. A first coupling is affixed to a distal-most end of the first robotic arm and a second coupling is affixed to a distal-most end of the second robotic arm. In various embodiments, the first coupling and the second coupling may be similar to the couplings described in more detail above and below.

In various embodiments, first position data may be collected for the distal end of the first robotic arm. In various embodiments, second positional data may be collected for the distal end of the second robotic arm. In various embodiments, the first and second positional data may be collected continuously. In various embodiments, the first and second positional data may be collected by manually moving the first and second robotic arms while they are coupled together. Positional data/information, as used herein, may generally be defined as (X, Y, Z) in a three-dimensional coordinate system. In various embodiments, collecting more positional data will provide more accurate calibration map for the two or more robotic arms.

When the first and second robotic arms are coupled together via the first and second couplings, the distal ends of each robotic are may be a predetermined, fixed distance away from one another. The positional information of the distal ends of each robotic arm and the fixed distance between the two distal ends may be used to generate a calibration value so that the two robotic arms may work together on a task, for example, suturing or cutting tissue. In various embodiments, once the calibration value is determined, the calibration value may be applied to all future positional information that is collected from the first and second robotic arms.

In various embodiments, the calibration value may be a calibration matrix. In various embodiments, the calibration matrix may be determined via a least mean squares (LMS) algorithm. LMS is a method in the family of stochastic gradient methods where statistics are estimated continuously. Since statistics are estimated continuously, the LMS algorithm can adapt to changes in the signal statistics and thus is an adaptive filter. In general, LMS minimizes E{|e(n)|²} similar to steepest descent, but based on unknown statistics. In various embodiments, the LMS algorithm uses estimates of the autocorrelation matrix R and the cross-correlation vector p. If instantaneous estimates are chosen, then the resulting method is the LMS algorithm shown in Equations 1a, 1b:

\hat{R}(n)=u(n)u^{H}(n) (Eqn. 1a)

\hat{p}(n)=u(n)d*(n) (Eqn. 1b)

In various embodiments, the calibration matrix may be determined via a least squares algorithm. In this method, a least squares solution to A [symbol] = [symbol] is an actual solution to A [symbol] = [symbol] _{∥}. The least squares algorithm minimizes | [symbol] −A [symbol] |²=Σ( [symbol] −A [symbol] )ᵢ². Equation 2a-2c shows the equation to solve for the least square solution:

A [symbol] = [symbol] _{∥}= [symbol] − [symbol] (Eqn. 2a)

A^{T}A [symbol] =A^{T} [symbol] −A^{T} [symbol] (Eqn. 2b)

A^{T}A [symbol] =A^{T} [symbol] (Eqn. 2c)

For example, a matrix A of positional data for the first robotic arm may be related to a matrix B of positional data for the second robotic arm by the following equation:

[A][H]=[B] (Eqn. 3a)

In Equations 3a-3c, A represents a matrix of three-dimensional positional data of the first robotic arm, H represents the calibration matrix, and B represents a matrix of three-dimensional positional data of the second robotic arm. The first row of matrix A may correspond to a first three-dimensional positional data point for the first robotic arm and the first row of matrix B may correspond to a first three-dimensional data point for the second robotic arm at the same sampling time. Each additional row in each matrix A, B may be an additional three-dimensional positional data point at another sampling time. To find the calibration matrix H,

[A]^{T}[A][H]=[A]^{T}[B] (Eqn. 3b)

[H]=([A]^{T}[A])⁻¹([A]^{T}[B]) (Eqn. 3c)

In various embodiments, a Kalman filter may be used to determine the calibration matrix.

Various embodiments may be adapted for use in gastrointestinal (GI) catheters, such as an endoscope. In particular, the endoscope may include an atomized sprayer, an IR source, a camera system and optics, a robotic arm, and an image processor.

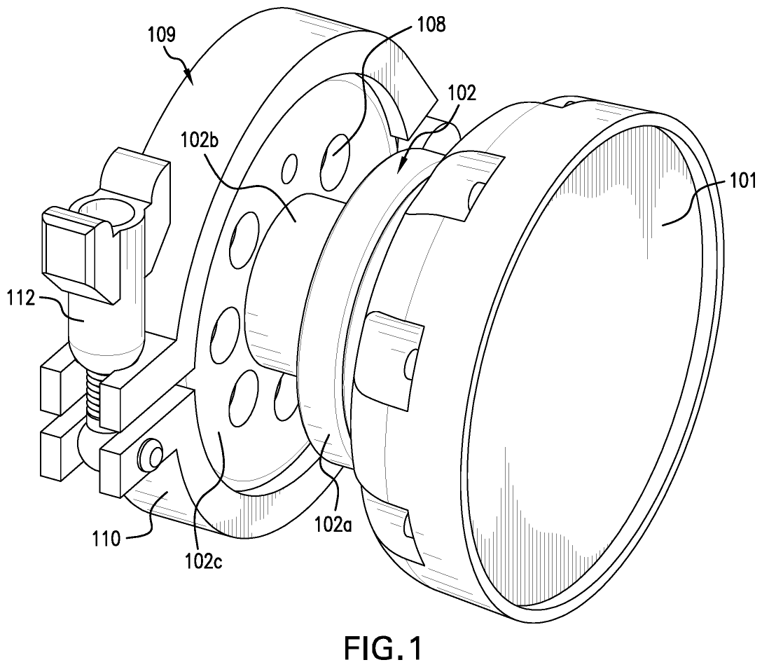

FIG. 1 illustrates an exemplary coupling 102 for a fixed-base robotic arm according to an embodiment of the present disclosure. The coupling 102 is attached to a distal-most end of a robotic arm 101. In particular, the coupling 102 includes a first flange 102a that is in contact with the robotic arm 101, a neck 102b extending from the first flange 102a, and a second flange 102c. In various embodiments, the second flange 102c may include one or more apertures 108 disposed in any suitable arrangement around the second flange 102c. The apertures 108 may be the same size or different sizes. The second flange 102c of the coupling 102 may further include one or more projection (or one or more recess) to thereby matingly engage one or more recess (or one or more projection) of a flange on a second coupling (not shown). A locking mechanism 109 may be used to thereby reversibly lock a first coupling 102 on a first robotic arm 101 to the second coupling on a second robotic arm during the calibration process. The locking mechanism 109 may include, for example, a collar 110 and a wing nut screw 112 used to force the two pieces of the collar together.

In various embodiments, after the first coupling 102 is locked to the second coupling 104, three-dimensional coordinate information may be recorded for each robotic arm while the robotic arms are stationary. In various embodiments, after the first coupling 102 is locked to the second coupling 104, the two couplings 102, 104 may be moved (e.g., manually by a user) together through three-dimensional space and three-dimensional coordinate information may be recorded for each robotic arm during the motion.

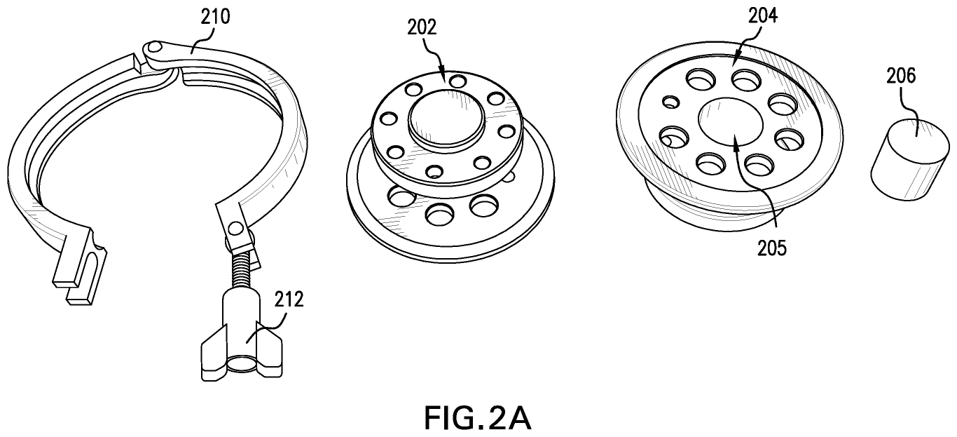

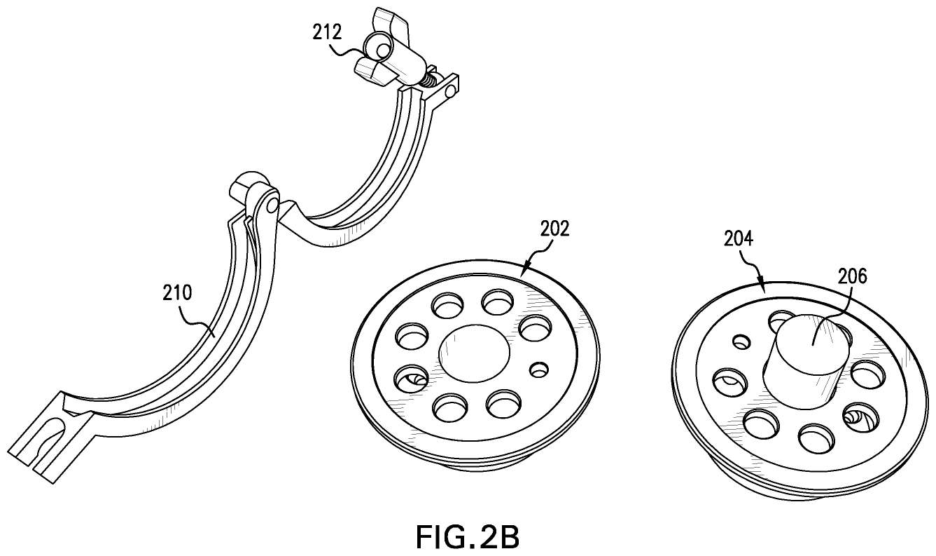

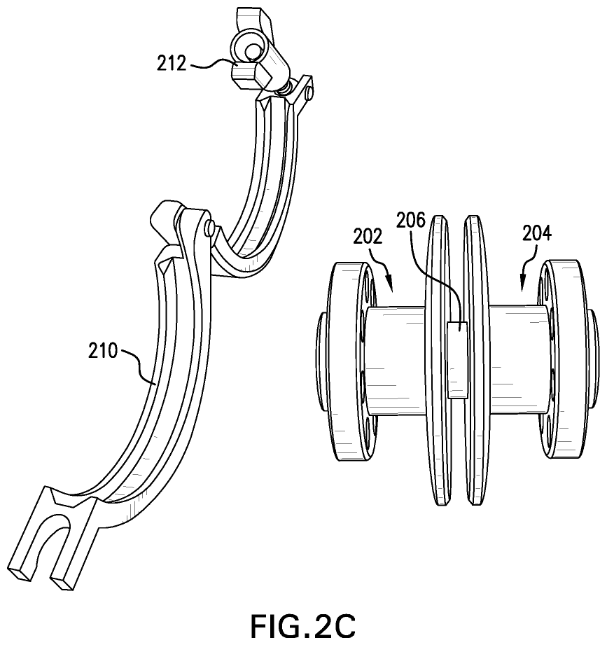

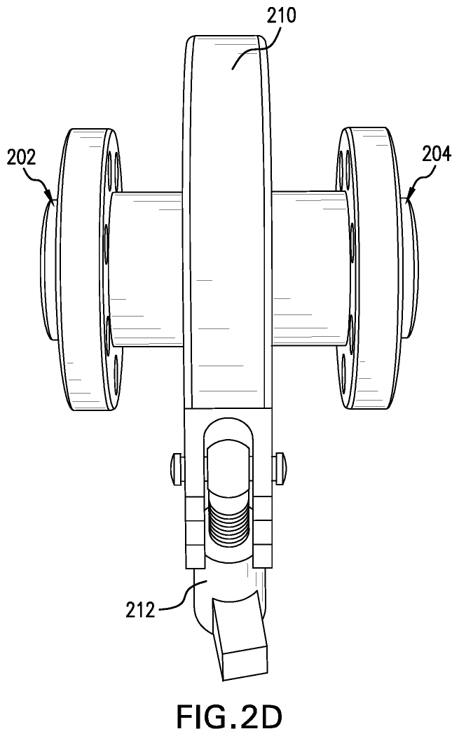

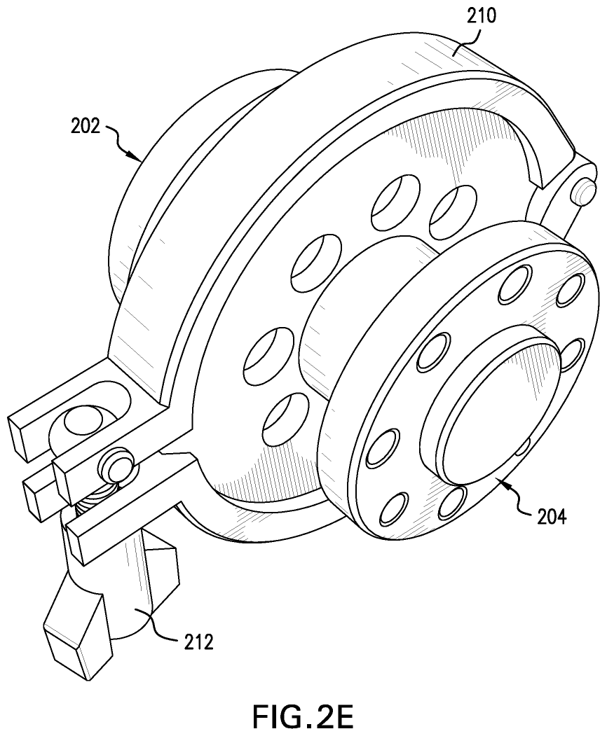

FIGS. 2A-2E illustrate steps to couple two exemplary couplings 202, 204 for fixed-base robotic arms together according to an embodiment of the present disclosure. One or both couplings 202, 204 may be substantially similar to the coupling described in FIG. 1. Both couplings 202, 204 include recesses 205 in which a removable pin 206 may be inserted to thereby couple the first coupling 202 with the second coupling 204. A locking mechanism having a collar 210 and a fastener 212 (e.g., wing nut screw) may be used to affix the first and second couplings 202, 204. FIG. 2A shows the couplings 202, 204, removable pin 206, and locking mechanism with collar 210 and fastener 212.

In various embodiments, a fastener as described above may be any suitable device or mechanism that is configured to hold the locking mechanism against the two couplings during calibration. In various embodiments, the fastener may include, for example, a screw, clamp, magnet, clasp, clip, pin, tie, wire, etc.

FIG. 2B shows the removable pin 206 inserted into the recess of the second coupling 204. FIG. 2C shows the first coupling 202 and the second coupling 204 coupled together by the removable pin 206. FIG. 2D shows the collar 210 placed around the flange of the first coupling 202 and the flange of the second coupling 204. FIG. 2E shows the fastener 212 tightened to thereby couple the couplings 202, 204 and prevent relative motion between the two couplings 202, 204.

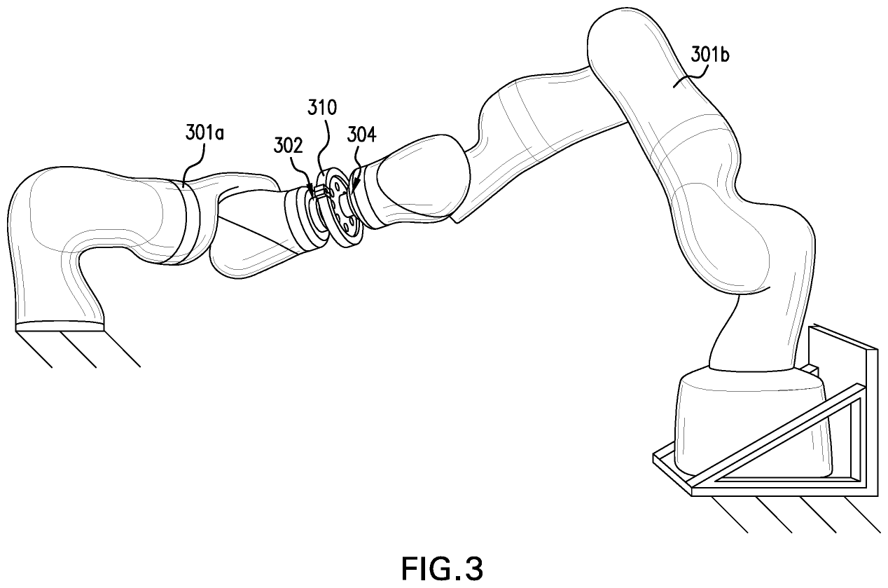

FIG. 3 illustrates two exemplary fixed-base robotic arms 301a, 301b coupled to one another according to an embodiment of the present disclosure. In FIG. 3, the first robotic arm 301a has a first coupling 302 and the second robotic arm 301b has a second coupling 304. The couplings 302, 304 are substantially similar to the couplings described above and are fixedly coupled to one another via a locking mechanism having a collar 310 and a wingnut screw (not shown). Once the robotic arms 301a, 301b are coupled to one another, a calibration matrix may be determined via the method described above. For example, while coupled together, the robotic arms 301a, 301b may be manually moved in various directions and for various distances to collect positional data for the distal ends of each robotic arm 301a, 301b.

In various embodiments, once the distal ends of each of the first and second robotic arms 301a, 301b are coupled together at a first coupled position, positional data may be recorded for the distal end of each robotic arm 301a, 301b (e.g., first positional data corresponding to the distal end of the first robotic arm and second positional data corresponding to the distal end of the second robotic arm). In various embodiments, a calibration value (e.g., calibration matrix) may be computed from this positional data. In various embodiments, the coupled robotic arms 301a, 301b may be moved in three-dimensional space to a second coupled position (e.g., a coupled position different from the first coupled position). In various embodiments, additional positional data may be recorded for each of the distal ends of the robotic arms 301a, 301b once the robotic arms 301a, 301b are in the second coupled position (e.g., third positional data corresponding to the distal end of the first robotic arm and fourth positional data corresponding to the distal end of the second robotic arm). In various embodiments, a calibration value (e.g., calibration matrix) may be computed from the recorded positional data at the second and/or first coupled position. In various embodiments, the coupled robotic arms 301a, 301b may be moved in three-dimensional space to a third coupled position. In various embodiments, additional positional data may be recorded once the robotic arms 301a, 301b are in the third coupled position (e.g., fifth positional data corresponding to the distal end of the first robotic arm and sixth positional data corresponding to the distal end of the second robotic arm). In various embodiments, a calibration value (e.g., calibration matrix) may be computed from the recorded positional data at the third, second and/or first coupled position. This process of moving the coupled distal ends to additional coupled positions and collecting positional data for each distal end may be repeated any suitable number of times to generate an accurate calibration value for each of the robotic arms 301a, 301b. In various embodiments, any combination (e.g., all or only a portion) of the recorded positional data may be used to compute the calibration value.

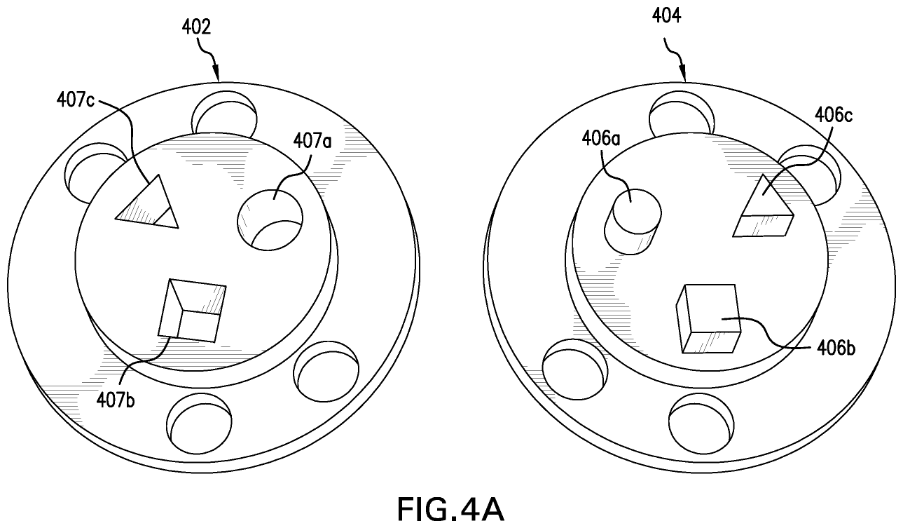

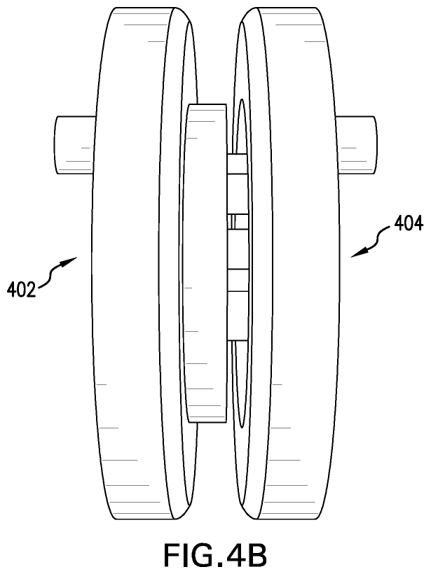

FIGS. 4A-4B illustrate two exemplary couplings 402, 404 for fixed-base robotic arms according to an embodiment of the present disclosure. FIG. 4A shows a first coupling 402 having recesses 407a-407c, where each recess has a different shape. In particular, recess 407a has a circular-shaped opening, and thus will only engage a pin having a circular cross-section, recess 407b has a square-shaped opening, and thus will only engage a pin having a square cross-section, and recess 407c has a triangular-shaped opening, and thus will only engage a pin having a triangular cross-section. FIG. 4A shows a second coupling 404 having pins 406a-406c, where each pin has a different shape corresponding to the recesses 407a-407c of the first coupling 402. In particular, pin 406a has a circular-shaped cross-section, and thus will only engage a recess having a circular opening, pin 406b has a square-shaped cross-section, and thus will only engage a recess having a square opening, and pin 406c has a triangular-shaped cross-section, and thus will only engage a recess having a triangular opening. FIG. 4B shows the first coupling engaging the second coupling. One skilled in the art will recognize that any suitable shape may be used for each of the recesses and pins, such as, for example, a circular shape, a triangular shape, a square shape, a diamond shape, an ovular shape, a rectangular shape, a pentagonal shape, a hexagonal shape, or a star shape.

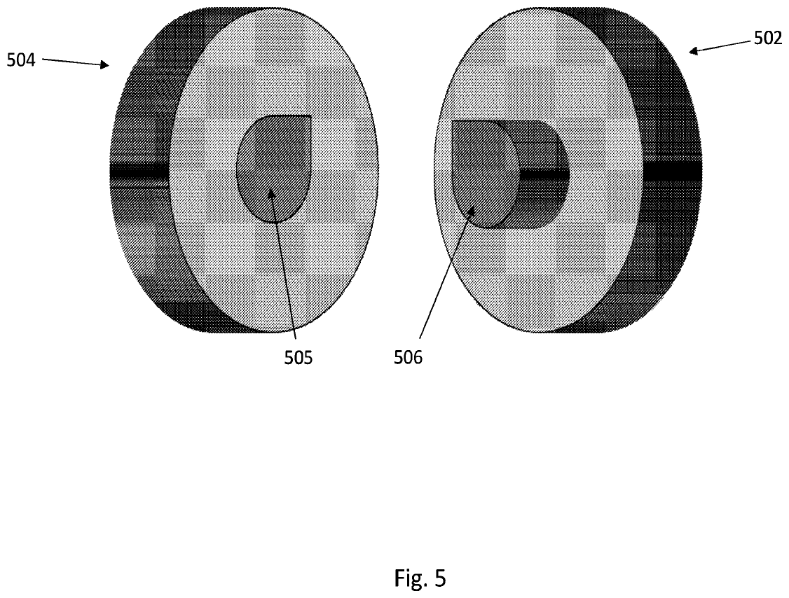

FIG. 5 illustrates exemplary couplings 502, 504 for a fixed-base robotic arm according to an embodiment of the present disclosure. In various embodiments, the first coupling 502 may include a protrusion 506 extending therefrom having a particular shape that corresponds to a recess 505 in the second coupling 504. In various embodiments, the protrusion 506 may have a shape that is asymmetric about one or more axes such that the protrusion 506 may only matingly engage the recess 505 in one particular orientation. For example, the protrusion 506 and the recess 505 may have a teardrop shape. In various embodiments, during calibration with couplings having an asymmetric shape, such as the teardrop shape, relative rotation between the couplings may be prevented such that the two robotic arms may be calibrated for both position and rotation. In various embodiments, the shape of the protrusion and recess have rotational symmetry about an axis normal to the respective flange.

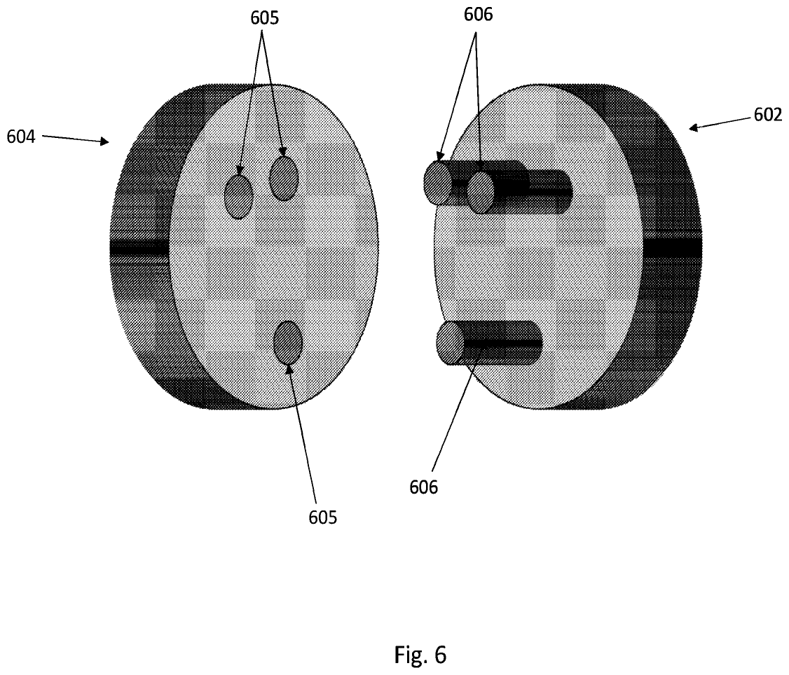

FIG. 6 illustrates exemplary couplings 602, 604 for a fixed-base robotic arm according to an embodiment of the present disclosure. In various embodiments, the first coupling 602 may include two or more protrusions 606 extending therefrom having a particular shape that corresponds to recesses 605 in the second coupling 604. In various embodiments, the protrusion 606 may have a shape that is symmetric about one or more axes (e.g., cylindrical). In various embodiments, the two or more protrusions may have a particular arrangement that allows coupling in a predetermined orientation. For example, two protrusions 606 are located at the top portion of the coupling 602 and one protrusion is located at the bottom of the coupling 602. Similarly, coupling 604 has two recesses 605 located at the top portion of the coupling 604 and one recess 605 located at the bottom of the coupling 604. In various embodiments, during calibration with couplings having an arrangement of protrusions/recesses that allow for coupling in a predetermined orientation, relative rotation between the couplings may be prevented such that the two robotic arms may be calibrated for both position and rotation.

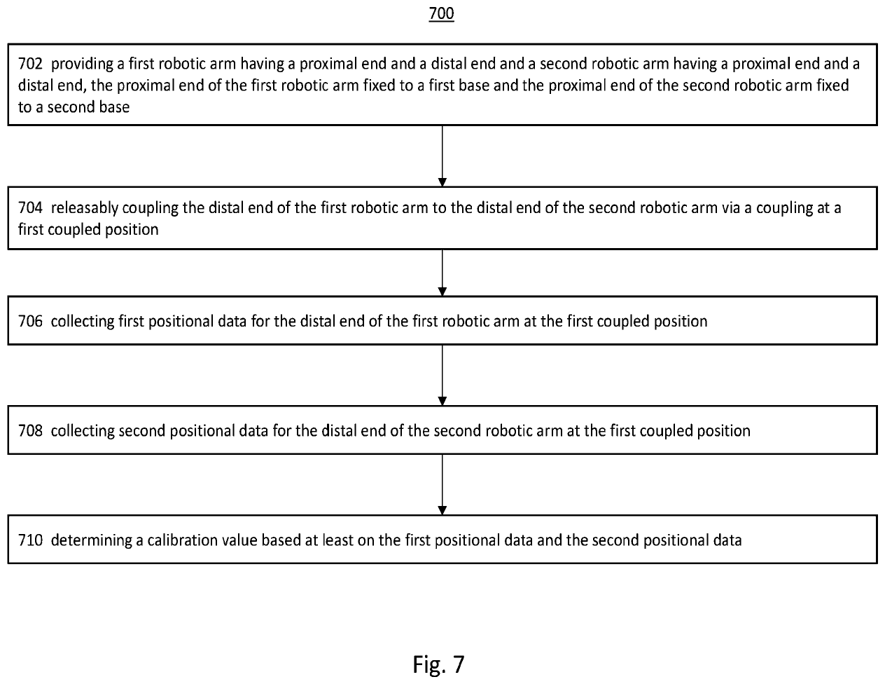

FIG. 7 shows a flowchart 700 of a method for calibrating two fixed-base robotic arms. At 702, a first robotic arm having a proximal end and a distal end and a second robotic arm having a proximal end and a distal end are provided. The proximal end of the first robotic arm is fixed to a first base and the proximal end of the second robotic arm is fixed to a second base. At 704, the distal end of the first robotic arm is releasably coupled to the distal end of the second robotic arm. At 706, first positional data for the distal end of the first robotic arm is collected at the first coupled position. At 708, second positional data for the distal end of the second robotic arm is collected at the first coupled position. At 710, a calibration value based at least on the first positional data and the second positional data is determined.

In various embodiments, the coupled distal ends of the robotic arms may be moved to a second coupled position. In various embodiments, third positional data for the distal end of the first robotic arm and fourth positional data for the distal end of the second robotic arm are collected at the first coupled position. In various embodiments, the calibration value is determined using the first, second, third, and fourth positional data. In various embodiments, the calibration value determined from the first and second positional data is applied to the third and fourth positional data.

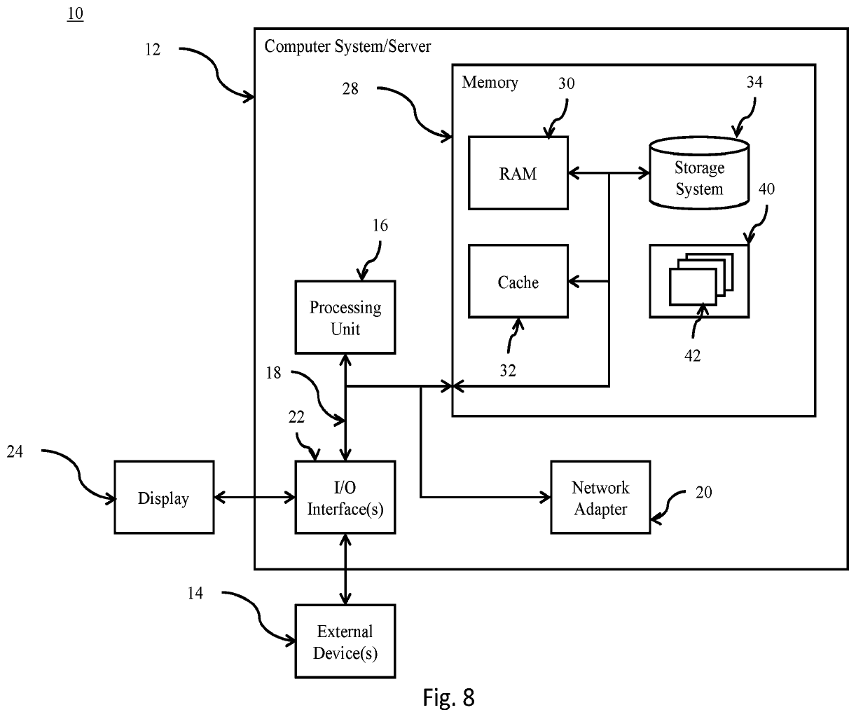

Referring now to FIG. 8, a schematic of an exemplary computing node is shown that may be used with the computer vision systems described herein. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 8, computer system/server 12 in computing node 10 is shown in the form of a general-purpose computing device. The components of computer system/server 12 may include, but are not limited to, one or more processors or processing units 16, a system memory 28, and a bus 18 coupling various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

In other embodiments, the computer system/server may be connected to one or more cameras (e.g., digital cameras, light-field cameras) or other imaging/sensing devices (e.g., infrared cameras or sensors).

The present disclosure includes a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In various embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In various alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-35. (canceled)

36. A method for controlling a plurality of robotic arms, the method comprising:
(a) providing two or more robotics arms comprising at least (i) a first robotic arm having a proximal end and a distal end and (ii) a second robotic arm having a proximal end and a distal end, the proximal end of the first robotic arm fixed to a first base and the proximal end of the second robotic arm fixed to a second base;
(b) releasably coupling the distal end of the first robotic arm to the distal end of the second robotic arm via a coupling at a first coupled position;
(c) collecting (i) first positional data for the distal end of the first robotic arm at the first coupled position and (ii) second positional data for the distal end of the second robotic arm at the first coupled position, wherein collecting the first positional data and the second positional data each comprises continuous recording of data; and
(d) determining a calibration value based at least in part on the first positional data and the second positional data.

37. The method of claim 36, further comprising moving the first and second robotic arms to a second coupled position after collecting the first positional data and the second positional data.

38. The method of claim 37, further comprising, subsequent to (c), collecting (iii) third positional data for the distal end of the first robotic arm while in the second coupled position and (iv) fourth positional data for the distal end of the second robotic arm while in the second coupled position.

39. The method of claim 38, wherein (d) further comprises determining or updating the calibration value based at least in part on the third positional data and the fourth positional data.

40. The method of claim 36, further comprising, subsequent to (d), applying the calibration value to adjust a position or an orientation of at least one of the first robotic arm and the second robotic arm.

41. The method of claim 38, further comprising applying the calibration value to each of the third positional data and the fourth positional data.

42. The method of claim 36, wherein the calibration value comprises a calibration matrix.

43. The method of claim 42, wherein the calibration matrix is determined using a least mean squares analysis.

44. The method of claim 42, wherein the calibration matrix is determined using a least squares analysis.

45. The method of claim 42, wherein the calibration matrix is determined using a Kalman filter.

46. The method of claim 36, further comprising, subsequent to (d), controlling a position, an orientation, or a movement of the first robotic arm and the second robotic arm to cooperatively perform a medical procedure.

47. The method of claim 46, wherein the position, the orientation, or the movement of the first robotic arm and the second robotic arm is adjusted or controlled based on the calibration value.

48. The method of claim 46, wherein the medical procedure comprises a gastric bypass surgery or a procedure involving a biological material selected from the group consisting of esophageal tissue, stomach tissue, small or large intestinal tissue, muscular tissue, dermal tissue, and internal organ tissue.

49. The method of claim 36, wherein the first positional data and the second positional data each comprise three-dimensional positioning data.

50. The method of claim 36, further comprising, subsequent to (d), moving the first and second robotic arms to one or more additional coupled positions and collecting additional positional data for each distal end of the first and second robotic arms at each additional coupled position.

51. The method of claim 50, further comprising updating the calibration value based at least in part on the additional positional data.

52. The method of claim 36, further comprising, subsequent to (d), using the first and second robotic arms to perform a surgical maneuver, wherein the surgical maneuver comprises at least one of (i) transferring an object between the first robotic arm and the second robotic arm, (ii) suturing, and (iii) cutting tissue.

53. The method of claim 36, further comprising, prior to (b), affixing a first coupling to the first robotic arm and a second coupling to the second robotic arm, wherein the first coupling comprises a set of protrusions and wherein the second coupling comprises a set of recesses corresponding to the set of protrusions.

54. The method of claim 53, wherein (b) further comprises moving the distal ends of the first robotic arm and the second robotic arm together to place the set of protrusions into the set of recesses to prevent a relative motion between the distal ends of the first and second robotic arms.

55. The method of claim 36, wherein the first and second positional data is collected (i) during a movement of the first and second robotic arms while the first and second robotic arms are coupled to each other or (ii) while the first and second robotic arms are stationary.
